SEQUENCE LISTING (9 sequences)

SEQ ID NO: 1 - protein, 147 aa, Homo sapiens
MVMGLGVLLLVFVLGLGLTPPTLAQDNSRYTHFLTQHYDAKPQGRDDRYCESIMRRRGLTSPCKDINTFIHGNKRSIKAICENKNGNPHRENLRISKSSFQVTTCKLHGGSPWPPCQYRATAGFRNVVVACENGLPVHLDQSIFRRP

SEQ ID NO: 2 - protein, 148 aa, Bos taurus
MVMVLSPLLLVFILGLGLTPVAPAQDDYRYIHFLTQHYDAKPKGRNDEYCFNMMKNRRLTRPCKDRNTFIHGNKNDIKAICEDRNGQPYRGDLRISKSEFQITICKHKGGSSRPPCRYGATEDSRVIVVGCENGLPVHFDESFITPRH

SEQ ID NO: 3 - protein, 145 aa, Mus musculus
MAISPGPLFLIFVLGLVVIPPTLAQDDSRYTKFLTQHHDAKPKGRDDRYCERMMKRRSLTSPCKDVNTFIHGNKSNIKAICGANGSPYRENLRMSKSPFQVTTCKHTGGSPRPPCQYRASAGFRHVVIACENGLPVHFDESFFSL

SEQ ID NO: 4 - protein, 139 aa, Gallus gallus
MAMSSLWWTAILLLALTVSMCYGVPTYQDFLRTHVDFPKTSFPNIAAYCNVMMVRRGINVHGRCKSLNTFVHTDPRNLNTLCINQPNRALRTTQQQLPVTDCKLIRSHPTCSYTGNQFNHRVRVGCWGGLPVHLDGTFP

SEQ ID NO: 5 - protein, 125 aa, Oryctolagus cuniculus
QDDSRYKHFLTQHYDAKPFGRNDRYCETMMKRRDLTSPCKDTNTFVHGNKGSIKDVCEDKNGKPYGKNFRISKSSFQVTTCKHVGGSPWPPCRYRATSGSRNIVIACENGLPVHFDESVFQQKVH

SEQ ID NO: 6 - protein, 123 aa, Sus scrofa
KDEDRYTHFLTQHYDAKPKGRDGRYCESIMKQRGLTRPCKEVNTFIHGTRNDIKAICNDKNGEPYNNFRRSKSPFQITTCKHKGGSNRPPCGYRATAGFRTIAVACENGLPVHFDESFIITSQ

SEQ ID NO: 7 - protein, 146 aa, Equus caballus
MAMSLCPLLLVFVLGLGLTPPSLAQDDSRYRQFLTKHYDANPRGRNDRYCESMMVRRHLTTPCKDTNTFIHGSKSSIKAICGNKNGNPYGETLRISKTRFQVTTCKHAGGSPRPPCRYRATPGFRSIVIACENGLPVHFDESFFRP

SEQ ID NO: 8 - DNA, 26 bp, Artificial Sequence
  Mutagenic primer
gtaatacacagaattcattccagaaa

SEQ ID NO: 9 - DNA, 22 bp, Artificial Sequence
  Mutagenic primer
tggataaaagacaggataactc

(19) United States
(12) Patent Application Publication
(10) Pub. No.: US 2013/0243728 A9
(43) Pub. Date: Sep. 19, 2013

(54) RECOMBINANT MICROORGANISMS

(71) Applicants: Matthew McDonagh, Williamstown (AU); Benjamin Cocks, Viewbank (AU); Angus Tester, Moonee Ponds (AU); Ross Crittenden, Moonee Ponds (AU)

(72) Inventors: Matthew McDonagh, Williamstown (AU); Benjamin Cocks, Viewbank (AU); Angus Tester, Moonee Ponds (AU); Ross Crittenden, Moonee Ponds (AU)

(73) Assignees: Agriculture Victoria Services PTY LTD, Attwood (AU); Murray Goulburn Co-Operative Co. Limited, Brunswick (AU)

(21) Appl. No.: 13/510,172

(22) PCT Filed: Nov. 18, 2010
(86) PCT No.: PCT/AU2010/001541

§ 371 Date: Oct. 18, 2012

(30) Foreign Application Priority Data
Nov. 18, 2009 (AU) 2009905638

(51) Int. Cl. C12N 15/63; C12N 1/19; A61K 35/74; A61P 21/00; C12P 21/00; C12N 1/21; A61K 38/17

(52) U.S. Cl. 424/93.2; 435/252.31; 435/252.32; 435/320.1; 435/254.23; 435/254.22; 435/254.21; 435/471; 514/1.1; 435/69.1; 514/20.9

(57) ABSTRACT

The invention relates to a recombinant microorganism comprising a transgene encoding angiogenin and optionally follistatin, a food product, beverage product or animal feed produced from or comprising said microorganism and uses thereof.

### FIELD

The present invention is in the field of recombinant microorganisms.

### BACKGROUND

RNase5/angiogenin is a 14 kDa non-glycosylated secreted ribonuclease known to regulate capillary formation and neuron survival, with functional mutations in the protein being a cause of the neuromuscular disorder amyotrophic lateral sclerosis (ALS). RNase5/angiogenin regulates endothelial and epithelial cell functions and is required for neuronal cell survival. Recent evidence indicates angiogenin is required for cell growth in epithelial and endothelial cells and the effective activity of growth factors such as VEGF, EGF and FGF.

In our co-pending application PCT/AU2009/000603 we demonstrated that angiogenin increases muscle cell growth and differentiation in vitro, and significantly alleviates the potent inhibitory effects of myostatin on myoblasts. Angiogenin is enriched in colostrum and milk, secretions which evolved to promote health, growth and development of suckling mammals. When added to the feed of mice, angiogenin purified from bovine milk increased exercising muscle growth by 50% over a 4 week period. We demonstrated that angiogenin is bioavailable when administered orally in our co-pending application PCT/AU2009/000602.

The activity of angiogenin on muscle in vivo and muscle cells in vitro provides a new molecular mechanism for the positive regulation of muscle growth, a hypothesis for the observed ability of milk to increase muscle accretion, and novel therapeutic opportunities to regulate aberrant neuromuscular functions and other diseases or disorders in which inhibition of myostatin has previously been suggested.

As angiogenin has substantial potential as a therapeutic, nutraceutical or functional food it is desirable to investigate recombinant methods for its production in increased yield. Additionally, as angiogenin has been shown to increase muscle mass when administered orally without toxicity, it is desirable to provide food, including animal feed, comprising enhanced levels of angiogenin.

### SUMMARY

The present invention in a first aspect provides a recombinant microorganism that includes a transgene encoding angiogenin.

The recombinant microorganism may also include a transgene encoding follistatin or the recombinant microorganism may include a transgene encoding angiogenin and follistatin.

The transgene may comprise a fusion protein comprising angiogenin and a signal sequence for a secreted polypeptide or protein and may further comprise a specific protease cleavage site between the signal sequence and angiogenin. The transgene may comprise an epitope tag to facilitate isolation of angiogenin from the organism.

In an embodiment, the angiogenin is co-expressed with ribonuclease inhibitor to enhance angiogenin expression.

The microorganisms may be utilised as a source of angiogenin, for use in pharmaceuticals, neutraceuticals and functional foods, in production of food products, such as fermentative microorganisms used in the production of beverages such as beer, wine and cider, fermented milk products such as yoghurts and buttermilks, cheeses, probiotic foods, or fermented meat products such as salami, and baked goods such as breads, including sourdough breads, and as animal or fish feed.

The recognition by the inventors that angiogenin is orally bioavailable and is heat stable make possible the provision of angiogenin in a food or beverage product by recombinant DNA methods, thus providing a reliable source of sufficient quantities of angiogenin in a convenient, inexpensive form for use in various prophylactic and therapeutic applications or for food or animal feed, including food for aquaculture, without the necessity for costly purification procedures.

In many embodiments, the transgene is chromosomally integrated. In many embodiments, the transgene includes a coding sequence for angiogenin, operably linked to a promoter.

The invention further provides an expression cassette comprising a coding sequence for angiogenin operably linked to a heterologous promoter. In many embodiments, the expression cassette is present in a vector.

The invention further provides a host cell, transformed with the expression cassette of the invention.

The invention further provides a method for producing a recombinant microorganism comprising an angiogenin transgene. The method generally involves introducing an angiogenin transgene into a microorganism by methods known to persons skilled in the art and culturing the microorganism in growth medium.

In one embodiment the microorganism secretes the angiogenin into the growth medium.

In another embodiment the microorganism does not secrete the angiogenin.

In one embodiment, the microorganism is a yeast, such as a yeast of a genus such as Candida, Debaromyces, Saccharomyces, Pichia, Hansenula, Kluyveromyces or the like, or Saccharomyces cerevisiae or Pichia pastoris. Where the yeast is Saccharomyces cerevisiae, it may be of a strain suitable for production of beer, wine, or bread.

In another embodiment the microorganism is a lactic acid bacterium or a probiotic organism. The lactic acid bacterium may be of the homolactic or heterolactic fermentation type.

In this embodiment the microorganism may be selected from the group consisting of Lactobacillus species, such as acidophilus, amylovorus, brevis, bulgaricus, buchneri, casei, confusus, crispatus, cucumeris, curvatis, delbrueckii, farciminis, fermentum, fructivorans, gasseri, helveticus, hilgardii, johnsonii, kefiri, lactis, leichmanii, lotus, paracasei, pasterianus, pentosus, plantarum, rhamnosus, reuteri, pentoaceticus, plantarum, sakei, salivarus, sanfranciscensis; Bifidobacterium species, such as adolescentis, animalis, bifidum, infantis, lactis, longum, pseudolongum and breve; Leuconostoc species, such as cremoris, lactis, mesenteroides and mesenteroides var. Sake, and onei; Micrococcus species; Pediococcus species, such as acidilatici, cerevisiae, halophilus, homari, pentosaceus and soyae; Propionibacterium species, such as acidipropioici, arabinosum, freudenreichii, shermani and thoenii; Acetobacter species, such as rancens and xylium; Bacillus species, such as brassicae fermentati, citreus, laterosporus, coagulans, licheniformis, natto and pumillus; Clostridium species, such as bifermentans; Corynebacterium species, such as kusaya, Halobacterium species, Halococcus species, Actococcus species, such as lactis and cremoris; Enterococcus species, such as durans and faecium; Enterobacter aerogenes; Staphylococcus species, such as aureus, carnosus, equorum, sciuri, xylosus, epidermidis, and Streptococcus species, such as cremoris, lactis, lactis var. diacetylactis, lactis var. hollandicus faecalis, and thermophilus.

Persons skilled in the art will be aware that although some of these organisms, such as Streptococcus faecalis, Staphylococcus aureus, and Staphylococcus epidermidis, are pathogens in certain situations, these organisms do have strains which are used in the food industry.

In another embodiment the microorgansim is a fungus, such as Penicillium camemberti, Penicillium roqueforti, Rhizopus oligosporus, Aspergillus oryzae or Monascus purpureus.

In another embodiment the microorganism is an alga, such as Spirulina (Arthrospira platensis or Arthrospira maxima), Chlorella spp. or Dunaliella salina. The microorganism itself, or its culture media, spores or live or processed versions of the recombinant microorganism may be utilised. Continuous harvest systems may be used. The microorganism, if for administering in food, or as animal or fish food, may be provided in a powder, tablet or capsule and may comprise a lyophilised, desiccated or spray dried culture of the microorganisms.

The invention further provides a method of production of a fermented food, feed, or beverage product, comprising the step of fermenting a culture comprising the recombinant microorganism or expression cassette of the invention.

The invention further provides the use of a recombinant microorganism comprising an angiogenin transgene in the production of a food product, beverage product or animal feed or as a source of recombinant angiogenin.

The food product may be baked goods (as angiogenin is heat stable), cheese, fermented milk, yoghurt, a fermented food product, or a probiotic food.

Fermented food products include beer, wine, bread, cheese, and yoghurt. Beer, wine, and bread production primarily uses yeasts, the most common of which is Saccharomyces cerevisiae. The production of certain types of bread, usually known as sour-dough breads, fermented meat products such as salami, fermented vegetable products and pulses, and milk products such as buttermilk, cheese and yoghurt, utilises bacterial starter cultures, which are most commonly members of the genus Lactobacillus. Organisms of the genus Leuconostoc are used in the production of foods such as sauerkraut, and in butter manufacture. In addition to this, certain wine styles are produced using malolactic fermentation, which also requires bacterial starter cultures, usually of the genera Lactobacillus, Leuconostoc or Pediococcus.

Although fermented foods have been used in a number of different human societies for many centuries, more recently it has been recognised that some of these foods, especially fermented milk products such as yoghurts, contain bacteria which are the same as or closely related to some of the bacteria which are normally present in the intestine and female genital tract, and that for this reason they are particularly easy to digest, and can even have a beneficial effect if the normal intestinal flora has been disrupted, for example because of antibiotic treatment for a medical condition. The bacteria present in these products are known as probiotic organisms, and foods containing them are referred to as probiotic foods. Such products are becoming increasingly popular, and for example the fermented milk product marketed under the name Yakult (Trade mark) is enormously popular in Japan, and has an increasing market in Australia.

The baking industry uses large quantities of defined yeast cultures to produce modern sour-dough breads and baked goods. Sour-dough bakery products are becoming increasingly popular as they stay soft longer, with less mould growth, than yeast-only breads. They are widely believed to be healthier, being more easily digested, and are regarded as being less likely to elicit allergies, and as being able to provide more vitamins, amino acids, and fatty acids than conventional bakery products. Examples of sour dough products include San Francisco-type sour-dough breads which are well known in San Francisco, U.S.A.; the organism used in production of this unique bread is Lactobacillus sanfrancisco, Panettone, Colombie, Pandoro, and various other small cakes and sweet baked goods widely produced in Italy, sour rye breads and baked goods such as pumpernickel, widely used in Germany, Scandinavia and other parts of northern Europe, Shamsy and Kisra breads of the Middle East.

The beverage product may be a beer, a wine or a cider.

The angiogenin may be recovered from the recombinant microorganism for addition to foods, may comprise the food itself or may be involved in production of the food.

For example, culture medium including angiogenin secreted from the recombinant microorganism of the invention may be added to products such as fruit juices or drinks, vegetable juices, or soft drinks.

The invention further provides a food or beverage product produced by or derived from the recombinant microorganism of the invention. The nature of the food product will depend on the nature of the expression host. The food product may further comprise follistatin.

It will be clearly understood that the invention also encompasses food or beverage products which are not themselves genetically modified, but which comprise angiogenin as defined above. Thus the invention further provides a food, beverage or food additive comprising angiogenin.

Other embodiments, which involve selectively enhancing expression of the endogenous angiogenin gene and optionally the endogenous follistatin gene in a recombinant microorganism, are contemplated.

The food product may be intended for human consumption or may be used as animal feed or in aquiculture. Consumption of such foods, optionally together with follistatin by animals increases the rate of growth of such animals, and increases the feed efficiency.

The invention further provides use of the subject recombinant microorganism as a source of angiogenin and optionally follistatin. Said angiogenin, optionally with follistatin may then be used for treatment of disease in animals, particularly humans. Diseases to be treated include those described in PCT/AU2009/000603, such as muscle disorders, including muscle wasting disorders, muscular dystrophy, muscular atrophy, sarcopenia, cachexia, improving muscle form by improving muscle strength, mass or exercise tolerance, decreasing fat, improving muscle to fat ratio, treating diseases caused by or involving suboptimal muscle to fat ratio which effect is enhanced by follistatin, treating bone disorders including osteoporosis, improving bone density, treating neurological disorders or diseases affecting the nervous system, particularly motor neurone diseases such as ALS, spinal muscular atrophys, inflammation myopathies including dermatomyositis, polymyositis and inclusion body myositis, diseases of the neuromuscular junction, such as Myasthenia Gravis (MG), Lambert-Eaton Syndrome (LES), and Congenital Myasthenic Syndrome (CMS), myopathies due to endocrine abnormalities, such as Hyperthyroid Myopathy (HYPTM) and Hypothyroid Myopathy (HYPOTM), diseases of peripheral nerve such as Charcot-Marie-Tooth Disease (CMT), Dejerine-Sottas Disease (DS), and Friedreich's Ataxia (FA), other myopathies including Myotonia Congenita (MC), Paramyotonia Congenita (PC), Central Core Disease (CCD), Nemaline Myopathy (NM), Myotubular Myopathy (MTM or MM), and

Periodic Paralysis (PP), wound healing, metabolic diseases of muscle, including Phosphorylase Deficiency (MPD or PYGM), Acid Maltase Deficiency (AMD), Phosphofructokinase Deficiency (PFKM), Debrancher Enzyme Deficiency (DBD), Mitochondrial Myopathy (MITO), Carnitine Deficiency (CD), Carnitine Palmityl Transferase Deficiency (CPT), Phosphoglycerate Kinase Deficiency (PGK), Phosphoglycerate Mutase Deficiency (PGAM or PGAMM), Lactate Dehydrogenase Deficiency (LDHA), and Myoadenylate Deaminase Deficiency (MAD), diseases connected to impaired lipid metabolism such as dyslipidemia and related lipid abnormalities such as hyperlipidemia, hypercholesteremia, hypertriglyceridemia and mixed dyslipidemia, spine injuries or diseases, diseases involving glucose homeostasis, for providing neuroprotection, nervous system functional support and managing metabolic diseases and diseases connected to impaired glucose metabolism and impaired insulin action including diabetes mellitus, especially diabetes mellitus type 1 and 2, non-autoimmune non-insulin dependent diabetes mellitus, syndrome X, metabolic syndrome or for improving gut health.

The present invention further provides a method for treating diseases as described above comprising the step of administering a food or beverage product according to the invention as part of the diet of an animal in need of such treatment. In one preferred embodiment, this aspect of the invention provides a method of improving the fat/lean ratio in domestic livestock raised for meat production.

It will be clearly understood that the method of the invention may be used in conjunction with one or more other such methods, including but not limited to dietary restriction or modification, exercise regimens, and administration of other modifiers of muscle or lipid metabolism.

The methods of the invention are applicable not only to humans, but also to other animals such as cattle, sheep, goats, pigs and horses, poultry animals such as chickens, geese and turkeys, companion animals such as cats and dogs, and zoo animals including felids, canids, and non-human primates. In particular, it will be appreciated that in domestic animals used for meat production, control of food utilisation so as to maximise lean body mass is generally considered to be desirable.

The methods of invention are also applicable to aquaculture to provide increased feed conversion to aquatic animals, such as fish, molluscs and shellfish, which is considered desirable.

The present invention further provides for use of the subject recombinant microorganism as a model for studying diseases involving angiogenin dysfunction and for identifying modulators of angiogenin and potential therapeutic candidates.

### DETAILED DESCRIPTION

The present invention provides recombinant microorganisms that include a transgene that encodes angiogenin and optionally follistatin and methods for producing such microorganisms.

Reference herein to recombinant microorganisms includes extracts of the recombinant microorganims, including live or dead microorganisms.

The subject recombinant microorganisms fall into at least two categories, depending on where the angiogenin is to be expressed:

1. Those secreting angiogenin into the culture media; and

2. Those which do not secrete angiogenin into the culture media.

Both types of recombinant microorganism can be used to provide a source of angiogenin for use in pharmaceuticals, neutraceuticals and functional foods or to provide food or animal feed.

Gram-positive bacteria such as lactobacillus, lactococcus, bifidobacteria and bacillus provide a “natural encapsulation” in the form of a cell wall for appropriate extended gut passage and digestive tract release of angiogenin for intestinal uptake.

Subject recombinant microorganisms have increased levels of angiogenin. In our co-pending applications PCT/AU2009/000602 and PCT/AU2009/000603 we demonstrate that angiogenin has an effect on muscle metabolism by oral administration and propose that angiogenin can be useful in treating muscle disorders, including muscle wasting disorders, muscular dystrophy, muscular atrophy, sarcopenia, cachexia, improving muscle form by improving muscle strength, mass or exercise tolerance, decreasing fat, improving muscle to fat ratio, treating diseases caused by or involving suboptimal muscle to fat ratio which effect is enhanced by follistatin, treating bone disorders including osteoporosis, improving bone density, treating neurological disorders or diseases affecting the nervous system, particularly motor neurone diseases such as ALS, spinal muscular atrophys, inflammation myopathies including dermatomyositis, polymyositis and inclusion body myositis, diseases of the neuromuscular junction, such as Myasthenia Gravis (MG), Lambert-Eaton Syndrome (LES), and Congenital Myasthenic Syndrome (CMS), myopathies due to endocrine abnormalities, such as Hyperthyroid Myopathy (HYPTM) and Hypothyroid Myopathy (HYPOTM), diseases of peripheral nerve such as Charcot-Marie-Tooth Disease (CMT), Dejerine-Sottas Disease (DS), and Friedreich's Ataxia (FA), other myopathies including Myotonia Congenita (MC), Paramyotonia Congenita (PC), Central Core Disease (CCD), Nemaline Myopathy (NM), Myotubular Myopathy (MTM or MM), and Periodic Paralysis (PP), wound healing, metabolic diseases of muscle, including Phosphorylase Deficiency (MPD or PYGM), Acid Maltase Deficiency (AMD), Phosphofructokinase Deficiency (PFKM), Debrancher Enzyme Deficiency (DBD), Mitochondrial Myopathy (MITO), Carnitine Deficiency (CD), Carnitine Palmityl Transferase Deficiency (CPT), Phosphoglycerate Kinase Deficiency (PGK), Phosphoglycerate Mutase Deficiency (PGAM or PGAMM), Lactate Dehydrogenase Deficiency (LDHA), and Myoadenylate Deaminase Deficiency (MAD), diseases connected to impaired lipid metabolism such as dyslipidemia and related lipid abnormalities such as hyperlipidemia, hypercholesteremia, hypertriglyceridemia and mixed dyslipidemia, spine injuries or diseases, diseases involving glucose homeostasis, for providing neuroprotection, nervous system functional support and managing metabolic diseases and diseases connected to impaired glucose metabolism and impaired insulin action including diabetes mellitus, especially diabetes mellitus type 1 and 2, non-autoimmune non-insulin dependent diabetes mellitus, syndrome X and metabolic syndrome and accordingly the food or feed products have potential to have such effects in animals, including humans which ingest the foods.

Angiogenin is involved in microbial inhibition, gut epithelial function, wound healing, and bacterial flora symbiosis and potentially ingesting foods containing angiogenin may have beneficial effects on gut health and gut based disease prevention and immune enhancement in humans and livestock animals, particularly when administered as a probiotic formulation or extract which can enable angiogenin to access gut tissues.

In the context of the invention described herein the main effect of angiogenin sought through the use of animal feed comprising the recombinant microorganism or their extracts in relation to livestock animals is the improvement of animal health including gut/immune function, and muscle mass and muscle to fat ratio to provide improved carcass composition. This is particularly important in livestock applications including for pigs, chickens (broilers and layers), beef, dairy, goats, sheep, shellfish and fish. Use of animal feed comprising recombinant microorganisms comprising an angiogenin transgene improves gut health and muscle growth and provides associated feed efficiency in livestock and can be used at all stages of development, including use of the microorganism in a milk replacement or supplement to enhance development. Angiogenin is expected to be a key regulator of high protein synthesis demand systems, such as gut epithelial cells, mammary gland epithelial cells producing milk proteins, and growing muscle.

Given the in vivo effects of oral angiogenin described in PCT/AU2009/000602 on mouse muscle and the role in regulating protein synthesis in muscle, administration of angiogenin would be expected to enhance muscle production in livestock animals. Given the conserved function of angiogenin in vertebrates in regulation of angiogenesis and activity when used across wide species boundaries, angiogenin is expected to have a role in the development of broiler chicken gut, immunity, muscle and growth and maintain health of chicken layers to enhance egg laying productivity.

For humans and companion animals, animal health and muscle composition can be improved and the above mentioned diseases can be treated or prevented by ingestion of the foods comprising recombinant angiogenin.

The recombinant microorganisms of the invention provide a ready source of angiogenin for use in pharmaceuticals, nutraceuticals and functional foods for treating or preventing the above mentioned diseases.

The following list defines terms, phrases and abbreviations used throughout the specification. Although the terms, phrases and abbreviations are listed in the singular tense, this list is intended to encompass all grammatical forms.

The term “transgene” is used herein to describe genetic material which has been or is about to be artificially inserted into the genome of a microorganism.

As used herein, the term “expression” includes transcription and translation.

As used herein, the term “heterologous” or “foreign” refers to nucleic acid and/or amino acid sequences not naturally occurring in the microorganism of interest. Heterologous sequences may also be found in a location or locations in the genome that differs from that in which it occurs in nature.

As used herein, the term “endogenous” refers to nucleic acid and/or amino acid sequences naturally occurring in the microorganism of interest.

As used herein, the term “recombinant” refers to genetic material, cells and/or microorganisms that have been genetically modified; for example, by addition of heterologous genetic material or modification of the endogenous genetic material.

As used herein, the term “isolated” or “purified” refers to nucleic acid and/or peptides or proteins that have been removed from at least one component with which it is naturally associated. For example, an isolated protein is substantially free of cellular material or culture medium when produced by molecular biological techniques.

As used herein, the term “vector” refers to a polynucleotide construct designed for transduction and/or transfection of one or more cell types.

As used herein, the phrase “operably linked” when referring to a transcriptional regulatory element and a coding sequence is intended to mean that the regulatory sequence is associated with the coding sequence in such a manner as to facilitate transcription of the coding sequence.

As used herein, the term “homologous recombination” refers to the exchange of DNA fragments between two DNA molecules or chromatids at the site of homologous nucleotide sequences.

As used herein, the term “gene targeting” refers to a type of homologous recombination that occurs when a fragment of genomic DNA is introduced into a cell and that fragment locates and recombines with endogenous homologous sequences.

As used herein, the term “hyperplasia” refers to an abnormal increase in the number of cells in an organ and/or tissue resulting in enlargement of the organ and/or tissue.

As used herein, the term “hypertrophy” refers to the enlargement of an organ and/or tissue resulting from an increase in the size of the individual cells of the organ and/or tissue.

As used herein, the term “genotype” refers to the entire genetic constitution of an organism; i.e. genes of an organism, both dominant and recessive.

As used herein, the term “phenotype” refers to the observable characteristics of an individual resulting from the interaction of the individual's genotype with the environment.

As used herein, the term “promoter” refers to a sequence at the 5′ end of a gene which binds DNA polymerase and/or transcription factors to regulate expression of the gene. Promoters can be tissue-specific.

The term “transformation” refers to a permanent or transient genetic change induced in a cell following the incorporation of new DNA (i.e. DNA exogenous to the cell).

The term “construct” refers to a recombinant nucleic acid, generally recombinant DNA, that has been generated for the purpose of the expression of a specific nucleotide sequence(s), or is to be used in the construction of other recombinant nucleotide sequences.

The term “cDNA” refers to all nucleic acids that share the arrangement of sequence elements found in native mature mRNA species, where sequence elements are exons and 3′ and 5′ non-coding regions. Normally mRNA species have contiguous exons, with the intervening introns removed by nuclear RNA splicing, to create a continuous open reading frame encoding the protein.

The term “genomic sequence” refers to a sequence having non-contiguous open reading frames, where introns interrupt the protein coding regions. It may further include the 3′ and 5′ untranslated regions found in the mature mRNA. It may further include specific transcriptional and translational regulatory sequences, such as promoters, enhancers, etc., including about 1 kb, but possibly more, of flanking genomic DNA at either the 5′ or 3′ end of the transcribed region. The genomic DNA may be isolated as a fragment of 100 kbp or smaller; and substantially free of flanking chromosomal sequence.

The invention in one aspect relates to the treatment of disorders. The terms “treating” and “treatment” as used herein refer to reduction in severity and/or frequency of symptoms, elimination of symptoms and/or underlying cause, prevention of the occurrence of symptoms (prophylaxis) and/or their underlying cause, and improvement or remediation of damage. Thus, for example, the present method of “treating” a disorder encompasses both prevention of the disorder in a predisposed individual and treatment of the disorder in a clinically symptomatic individual.

“Treating” as used herein covers any treatment of, or prevention of a condition in a vertebrate, a mammal, particularly a human, and includes: inhibiting the condition, i.e., arresting its development; or relieving or ameliorating the effects of the condition, i.e., cause regression of the effects of the condition.

“Prophylaxis” or “prophylactic” or “preventative” therapy as used herein includes preventing the condition from occurring or ameliorating the subsequent progression of the condition in a subject that may be predisposed to the condition, but has not yet been diagnosed as having it.

Angiogenin and follistatin as referred to herein, particularly with regard to transgenes and proteins/polypeptides, encompass full length angiogenin and follistatin from any mammalian species as well as functional fragments and analogues thereof. In one embodiment angiogenin is of human or bovine origin.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a”, “and”, and “the” include plural referents unless the context clearly dictates otherwise. Thus, for example, reference to “a transgenic non-human animal” includes a plurality of such animals and reference to “the transgene” includes reference to one or more transgenes and equivalents thereof known to those skilled in the art, and so forth.

In further describing the subject invention, the subject recombinant microorganisms and methods for their production are described first in greater detail, followed by a review of representative applications to which the subject microorganisms find use, e.g., in food production, etc.

### Recombinant Microorganisms and Methods for their Production

The present invention provides a recombinant microorganism that includes an angiogenin transgene. An angiogenin transgene includes a nucleotide sequence that encodes angiogenin. In many embodiments, the angiogenin coding sequence is operably linked to a promoter. As follistatin is considered to enhance angiogenin activity the recombinant microorganism or the angiogenin transgene may also include a follistatin transgene. The microorganism may also co-express angiogenin with ribonuclease inhibitor to enhance angiogenin expression levels.

The description provided herein as it relates to angiogenin transgenes and recombinant microorganisms is meant to be exemplary only, and is not meant to be limited to particular angiogenin transgenes and recombinant microorganisms. Any angiogenin transgene can be used to generate a subject recombinant microorganism, provided that the subject recombinant microorganism exhibits increased concentration of angiogenin when compared to a non-recombinant microorganism.

A transgene having a coding region for angiogenin is used to transform a cell, meaning that a permanent or transient genetic change, generally a permanent genetic change, is induced in a cell following incorporation of the exogenous DNA of the transgene. A permanent genetic change is generally achieved by introduction of the DNA into the genome of the cell. Vectors for stable integration include plasmids, retroviruses and other animal viruses, YACs, and the like.

Recombinant microorganisms of the invention comprise an exogenous nucleic acid sequence present as an extrachromosomal element or stably integrated therein. Unless otherwise indicated, it will be assumed that a recombinant microorganism comprises stable changes to the germline sequence.

In some embodiments, the angiogenin transgene that is introduced into the microorganism includes an exogenous angiogenin coding sequence. The exogenous gene is in some embodiments from a different species than the host (e.g., is a heterologous angiogenin gene). The exogenous gene may or may not be altered in its coding sequence. Non-coding sequences, such as control elements, may or may not be present. Control elements, if present in the transgene, include homologous (e.g., normally associated with the coding sequence) or heterologous (e.g., not normally associated with the coding region, e.g., from another species) control elements. The introduced gene may be a wild-type gene, naturally occurring polymorphism, or a genetically manipulated sequence, for example having deletions, substitutions or insertions in the coding or non-coding regions. The angiogenin coding region may be operably linked to a promoter, which may be constitutive or inducible, and other regulatory sequences required for expression and or secretion in the host microorganism. Alternatively, the angiogenin coding region may not be operably linked to a control element(s) in the transgene, but instead becomes operably linked to control element(s) when it becomes integrated into the genome.

In other embodiments, the endogenous angiogenin coding sequence is upregulated. In these embodiments, the angiogenin coding sequence may or may not be operably linked to control element(s). The angiogenin coding region may be operably linked to a promoter, which may be constitutive or inducible, and other regulatory sequences required for expression in the host microorganism. Alternatively, the angiogenin coding region may not be operably linked to a control element(s), but instead becomes operably linked to control element(s) when the transgene becomes integrated into the genome. For example gene editing using zinc finger transcription factors fused to endonucleases (Sangamo technology) may be a useful approach for upregulating endogenous angiogenin expression.

The angiogenin transgene may comprise angiogenin from any species but particularly includes from human, bovine, porcine, equine, avian, ovine, rat, chicken, turkey or mouse angiogenin. The transgene may encode angiogenin having SEQ ID NO: 1 (human), SEQ ID NO: 2 (bovine), SEQ ID NO: 3 (mouse), SEQ ID NO: 4 (chicken), SEQ ID NO: 5 (rabbit), SEQ ID NO: 6 (pig), SEQ ID NO: 7 (horse), or any other sequence encoding angiogenin or a functional fragment thereof capable of inducing growth of myoblasts in cell culture.

```
(SEQ ID NO: 1)
                10 20 30 40 50 60
MVMGLGVLLL VFVLGLGLTP PTLAQDNSRY THFLTQHYDA KPQGRDDRYC ESIMRRRGLT
                70 80 90 100 110 120
SPCKDINTFI HGNKRSIKAI CENKNGNPHR ENLRISKSSF QVTTCKLHGG SPWPPCQYRA
              130 140
TAGFRNVVVA CENGLPVHLD QSIFRRP
(SEQ ID NO: 2)
                10 20 30 40 50 60
MVMVLSPLLL VFILGLGLTP VAPAQDDYRY IHFLTQHYDA KPKGRNDEYC FNMMKNRRLT
                70 80 90 100 110 120
RPCKDRNTFI HGNKNDIKAI CEDRNGQPYR GDLRISKSEF QITICKHKGG SSRPPCRYGA
              130 140
TEDSRVIVVG CENGLPVHFD ESFITPRH
(SEQ ID NO: 3)
                10 20 30 40 50 60
MAISPGPLFL IFVLGLVVIP PTLAQDDSRY TKFLTQHHDA KPKGRDDRYC ERMMKRRSLT
                70 80 90 100 110 120
SPCKDVNTFI HGNKSNIKAI CGANGSPYRE NLRMSKSPFQ VTTCKHTGGS PRPPCQYRAS
              130 140
AGFRHVVIAC ENGLPVHFDE SFFSL
(SEQ ID NO: 4)
                10 20 30 40 50 60
MAMSSLWWTA ILLLALTVSM CYGVPTYQDF LRTHVDFPKT SFPNIAAYCN VMMVRRGINV
                70 80 90 100 110 120
HGRCKSLNTF VHTDPRNLNT LCINQPNRAL RTTQQQLPVT DCKLIRSHPT CSYTGNQFNH
              130
RVRVGCWGGL PVHLDGTFP
(SEQ ID NO: 5)
                10 20 30 40 50 60
QDDSRYKHFL TQHYDAKPFG RNDRYCETMM KRRDLTSPCK DTNTFVHGNK GSIKDVCEDK
                70 80 90 100 110 120
NGKPYGKNFR ISKSSFQVTT CKHVGGSPWP PCRYRATSGS RNIVIACENG LPVHFDESVF
QQKVH
(SEQ ID NO: 6)
                10 20 30 40 50 60
KDEDRYTHFL TQHYDAKPKG RDGRYCESIM KQRGLTRPCK EVNTFIHGTR NDIKAICNDK
                70 80 90 100 110 120
NGEPYNNFRR SKSPFQITTC KHKGGSNRPP CGYRATAGFR TIAVACENGL PVHFDESFII
TSQ
(SEQ ID NO: 7)
                10 20 30 40 50 60
MAMSLCPLLL VFVLGLGLTP PSLAQDDSRY RQFLTKHYDA NPRGRNDRYC ESMMVRRHLT
                70 80 90 100 110 120
TPCKDTNTFI HGSKSSIKAI CGNKNGNPYG ETLRISKTRF QVTTCKHAGG SPRPPCRYRA
              130 140
TPGFRSIVIA CENGLPVHFD ESFFRP
```

### Methods of Making a Subject Recombinant Microorganism

The invention provides methods of generating a subject recombinant microorganism. The method generally involves introducing an angiogenin transgene, into a microorganism such that the transgene is integrated into the genome of the microorganism and angiogenin is secreted into culture medium or maintained in the cytoplasm or periplasm. Any method of making recombinant microorganisms can be used as described, as will be well known to persons skilled in the art.

### Expression Vectors and Transgenes

A subject recombinant microorganism is typically generated by a method involving introducing into a cell a construct comprising a nucleotide sequence encoding angiogenin. An angiogenin transgene includes, at a minimum, a coding region for angiogenin. In some embodiments, the nucleotide sequence encoding angiogenin is operably linked to a promoter and, optionally, additional control elements, that provide for increased expression of the transgene in the microorganisms. In other embodiments, the nucleotide sequence encoding angiogenin is not operably linked to any control elements. Instead, the angiogenin transgene includes, on the 5′ and 3′ ends of the coding region, sequences that provide for homologous recombination with an endogenous gene.

As discussed above any angiogenin gene can be used in the transgene, including those encoding the angiogenin sequences provided as SEQ ID NO: 1 to 7. The transgene or recombinant microorganism may also comprise recombinant follistatin.

Sequences that vary from a known coding sequence for a given angiogenin can be used, as long as the encoded angiogenin has substantially the same activity in inducing growth of myoblasts in cell culture. For example, the encoded angiogenin can include one or more conservative amino acid substitutions compared to the amino acid sequence of a known angiogenin. Non-limiting examples of conservative amino acid substitutions are Phe/Tyr; Ala/Val; Leu/Ile; Arg/His; Ser/Thr; etc. The encoded angiogenin can also include insertions or deletions (including truncations) of one or more amino acid residues, compared to the amino acid sequence of a known angiogenin. Further, the encoded angiogenin can include one or more naturally occurring polymorphisms. The angiogenin coding sequence can be completely or partially synthetic. An angiogenin coding sequence can also be a consensus sequence, derived, e.g., by comparing the angiogenin coding sequences from two or more species, and deriving therefrom a consensus sequence, using standard methods. An optimised angiogenin sequence can also be used, for example a sequence that includes mutations that confer greater activity, more protease resistance, heat stability, cell wall treatment compatibility, etc.

Any known coding sequence for angiogenin can be used to make a subject recombinant microorganism, including an angiogenin coding sequence from mouse, human, cow, sheep, etc. The coding sequence can be a cDNA sequence, or a genomic sequence. The coding sequence for the angiogenin may be, but need not be, from the same species as the recombinant microorganism.

A suitable nucleotide sequence encoding angiogenin generally has at least about 70%, at least about 80%, at least about 85%, at least about 90%, at least about 95%, or at least about 98%, or higher, nucleotide sequence identity with a known coding sequence for angiogenin. Sequence similarity is calculated based on a reference sequence, which may be a subset of a larger sequence, such as a conserved motif, coding region, flanking region, etc. A reference sequence will usually be at least about 18 nucleotides long, more usually at least about 30 nucleotides long, and may extend to the complete sequence that is being compared. Algorithms for sequence analysis are known in the art, such as BLAST, described in Altschul et al. (1990), J. Mol. Biol. 215:403-10 (using default settings).

Also suitable for use are angiogenin coding sequences that hybridize under stringent hybridization conditions to a known angiogenin coding sequence. An example of stringent hybridization conditions is hybridization at 50° C. or higher and 0.1×SSC (15 mM sodium chloride/1.5 mM sodium citrate). Another example of stringent hybridization conditions is overnight incubation at 42° C. in a solution: 50% formamide, 1×SSC (150 mM NaCl, 15 mM sodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at about 65° C. For example, high stringency conditions include aqueous hybridization (e.g., free of formamide) in 6×SSC (where 20×SSC contains 3.0 M NaCl and 0.3 M sodium citrate), 1% sodium dodecyl sulfate (SDS) at 65° C. for about 8 hours (or more), followed by one or more washes in 0.2×SSC, 0.1% SDS at 65° C. For example, moderate stringency conditions include aqueous hybridization (e.g., free of formamide) in 6×SSC, 1% SDS at 65° C. for about 8 hours (or more), followed by one or more washes in 2×SSC, 0.1% SDS at room temperature.

As noted above, in some embodiments, an angiogenin transgene includes a coding sequence for angiogenin operably linked to one or more control sequences, e.g., promoters, 3′ transcriptional control sequences, translational control elements, etc.

In some embodiments, an angiogenin transgene is not operably linked to a control element. Instead, the transgene includes sequences that provide for homologous recombination with an endogenous gene, such that the angiogenin coding sequence replaces all or part of endogenous coding sequence, and the integrated angiogenin coding region is under transcriptional control of endogenous control element(s). For example, an angiogenin transgene includes 5′ and 3′ flanking sequences that are homologous to sequences in the 5′ and 3′ regions of a β-lactoglobulin gene, such that the transgene integrates into the genome of a cell by homologous recombination, whereby the angiogenin coding sequences of the transgene replace the endogenous β-lactoglobulin gene, and the angiogenin coding sequence integrates into the genome and is under the transcriptional control of the endogenous β-lactoglobulin control elements. Methods for carrying out homologous recombination are well known in the art.

An angiogenin transgene is generally provided as part of a vector (e.g., an angiogenin construct), a wide variety of which are known in the art and need not be elaborated upon herein. Vectors include, but are not limited to, plasmids; cosmids; viral vectors; artificial chromosomes (HACs, YACs, BACs, etc.); mini-chromosomes; and the like. Vectors are amply described in numerous publications well known to those in the art. Vectors provide for expression of the subject nucleic acids, may provide for propagating the subject nucleic acids, or both.

For expression, e.g., where the transgene includes a promoter, an expression cassette may be employed. The expression vector will provide a transcriptional and translational initiation region, which may be inducible or constitutive, where the coding region is operably linked under the transcriptional control of the transcriptional initiation region, and a transcriptional and translational termination region. These control regions may be native to the angiogenin gene, or may be derived from exogenous sources.

Where the transgene includes a promoter, an expression vector will generally have convenient restriction sites located near the promoter sequence to provide for the insertion of nucleic acid sequences encoding angiogenin. A selectable marker operative in the expression host may be present. Expression vectors may be used for the production of fusion proteins, where the exogenous fusion peptide provides additional functionality, i.e. increased protein synthesis, stability, reactivity with defined antisera, an enzyme marker, e.g. β-galactosidase, etc.

Expression cassettes may be prepared comprising a transcription initiation region, the gene or fragment thereof, and a transcriptional termination region.

The angiogenin sequence used may be modified to improve myogenic activity through improved RNase enzyme activity, improved ribosomal RNA transcriptional activation and/or DNA binding activity, improved ribosomal RNA processing/splicing activity and improved receptor binding and endocytosis.

Fusions of angiogenin at the N or C terminus are also contemplated, for example angiogenin single chain immunofusions.

The angiogenin sequence may be one in which a mutation to decrease RNase activity is included.

### Utility

The subject recombinant microorganisms find use in a variety of applications, including, but not limited to, food production, research, production of angiogenin and the like. For example, the subject microorganisms find use in producing food products that have higher angiogenin or provide greater muscle development than those produced naturally.

Such food products can be used as a source of angiogenin. The subject microorganisms find use in research, to analyse the effects of angiogenin and its proposed modulators in various tissues.

### Food Applications

The present invention provides recombinant microorganisms as a source of angiogenin, methods for producing food products from a subject recombinant microorganism, and food products harvested a non-human animal fed with the subject recombinant microorganism. Where the food product requires further processing, the methods involve harvesting a food product from a subject recombinant microorganism, and processing the food product. Thus, the invention provides a method of producing a processed food product, involving processing a food product harvested from a subject recombinant microorganism. The invention further provides a processed food product obtained by processing a food product harvested from a subject recombinant microorganism.

Methods of harvesting angiogenin from a subject recombinant microorganism are well known to those skilled in protein purification.

The present invention further provides food products produced by a subject recombinant microorganism, and processed food products made with such food products. Food products include any preparation for human consumption including for enteral or parenteral consumption, which when taken into the body (a) serve to nourish or build up tissues or supply energy and/or (b) maintain, restore or support adequate nutritional status or metabolic function.

Food products of the invention are suitable for consumption by any individual. As used herein, the term “individual” includes human and non-human individuals. Non-human individuals include animals, particularly mammals, e.g., farm animals, pets, etc. The recombinant microorganisms can be used as animal feed of for feed in aquaculture in appropriate circumstances.

A variety of beneficial effects are attributed to angiogenin, including increased muscle function and mass, improved fat to weight composition, exercise tolerance, and involvement in neuro-muscular disease such as ALS.

Angiogenin can also increase the growth rate of farm animals fed with a diet that includes angiogenin. Thus, a subject food product increases the growth rate and feed efficiency of a farm animal fed with a subject food product. Thus, a subject food product is of particular interest for feeding a farm animal (e.g., a pig, a cow, a goat, etc.).

The present invention provides food products, including nutraceutical formulations, which include angiogenin. The term “nutraceutical formulation” refers to a food or part of a food that offers medical and/or health benefits including prevention or treatment of disease. Nutraceutical products range from isolated nutrients, dietary supplements and diets, to genetically engineered designer foods, functional foods, herbal products and processed foods such as cereal, soup and beverages. The term “functional foods,” refers to foods that include “any modified food or food ingredients that may provide a health benefit beyond the traditional nutrients it contains.”

Nutraceutical formulations of interest include foods for veterinary or human use, including food bars (e.g. cereal bars, breakfast bars, energy bars, nutritional bars); chewing gums; drinks; fortified drinks; drink supplements (e.g., powders to be added to a drink); tablets; and the like.

A subject food product or nutraceutical formulation may include angiogenin and at least one additional food-grade component. Suitable components include, but are not limited to, mono- and disaccharides; carbohydrates; proteins; amino acids; fatty acids; lipids; stabilizers; preservatives; flavoring agents; coloring agents; sweeteners; antioxidants, chelators, and carriers; texturants; nutrients; pH adjusters; emulsifiers; stabilizers; milk base solids; edible fibers; and the like. The food component can be isolated from a natural source, or can be synthesized. All components are food-grade components fit for human consumption.

Examples of suitable monosaccharides include sorbitol, mannitol, erythrose, threose, ribose, arabinose, xylose, ribulose, glucose, galactose, mannose, fructose, and sorbose. Non-limiting examples of suitable disaccharides include sucrose, maltose, lactitol, maltitol, maltulose, and lactose.

Suitable carbohydrates include oligosaccharides, polysaccharides, and/or carbohydrate derivatives. As used herein, the term “oligosaccharide” refers to a digestible linear molecule having from 3 to 9 monosaccharide units, wherein the units are covalently connected via glycosidic bonds. As used herein, the term “polysaccharide” refers to a digestible (i.e., capable of metabolism by the human body) macromolecule having greater than 9 monosaccharide units, wherein the units are covalently connected via glycosidic bonds. The polysaccharides may be linear chains or branched. Carbohydrate derivatives, such as a polyhydric alcohol (e.g., glycerol), may also be utilized as a complex carbohydrate herein. As used herein, the term “digestible” in the context of carbohydrates refers to carbohydrate that are capable of metabolism by enzymes produced by the human body. Examples of polysaccharides that are non-digestible carbohydrates are cellulose, resistant starches (e.g., raw corn starches) and retrograded amyloses (e.g., high amylose corn starches). Non-limiting examples of carbohydrates include raffinoses, stachyoses, maltotrioses, maltotetraoses, glycogens, amyloses, amylopectins, polydextroses, and maltodextrins.

Suitable fats include, but are not limited to, triglycerides, including short-chain (C₂-C₄) and long-chain triglycerides (C₁₆-C₂₂).

Suitable texturants (also referred to as soluble fibers) include, but are not limited to, pectin (high ester, low ester); carrageenan; alginate (e.g., alginic acid, sodium alginate, potassium alginate, calcium alginate); guar gum; locust bean gum; psyllium; xanthan gum; gum arabic; fructo-oligosaccharides; inulin; agar; and functional blends of two or more of the foregoing.

Suitable emulsifiers include, but are not limited to, propylene glycol monostearate

(PGMS), sodium stearoyl lactylate (SSL), calcium stearoyl lactylate (CSL), monoglycerides, diglycerides, monodiglycerides, polyglycerol esters, lactic acid esters, polysorbate, sucrose esters, etc.

Edible fibers include polysaccharides, oligosaccharides, lignin and associated plant substances. Suitable edible fibers include, but are not limited to, sugar beet fiber, apple fiber, pea fiber, wheat fiber, oat fiber, barley fiber, rye fiber, rice fiber, potato fiber, tomato fiber, other plant non-starch polysaccharide fiber, and combinations thereof.

Suitable flavoring agents include natural and synthetic flavors, “brown flavorings” (e.g., coffee, tea); dairy flavorings; fruit flavors; vanilla flavoring; essences; extracts; oleoresins; juice and drink concentrates; flavor building blocks (e.g., delta lactones, ketones); and the like; and combinations of such flavors. Examples of botanic flavors include, for example, tea (e.g., preferably black and green tea), aloe vera, guarana, ginseng, ginkgo, hawthorn, hibiscus, rose hips, chamomile, peppermint, fennel, ginger, licorice, lotus seed, schizandra, saw palmetto, sarsaparilla, safflower, St. John's Wort, curcuma, cardamom, nutmeg, cassia bark, buchu, cinnamon, jasmine, haw, chrysanthemum, water chestnut, sugar cane, lychee, bamboo shoots, vanilla, coffee, and the like.

Suitable sweeteners include, but are not limited to, alitame; dextrose; fructose; lactilol; polydextrose; xylitol; xylose; aspartame, saccharine, cyclamates, acesulfame K, L-aspartyl-L-phenylalanine lower alkyl ester sweeteners, L-aspartyl-D-alanine amides; L-aspartyl-D-serine amides; L-aspartyl-hydroxymethyl alkane amide sweeteners; L-aspartyl-1-hydroxyethylalkane amide sweeteners; and the like.

Suitable anti-oxidants include, but are not limited to, tocopherols (natural, synthetic); ascorbyl palmitate; gallates; butylated hydroxyanisole (BHA); butylated hydroxytoluene (BHT); tert-butyl hydroquinone (TBHQ); and the like.

Suitable nutrients include vitamins and minerals, including, but not limited to, niacin, thiamin, folic acid, pantothenic acid, biotin, vitamin A, vitamin C, vitamin B₂, vitamin B₃, vitamin B₆, vitamin B₁₂, vitamin D, vitamin E, vitamin K, iron, zinc, copper, calcium, phosphorous, iodine, chromium, molybdenum, and fluoride.

Suitable coloring agents include, but are not limited to, FD&C dyes (e.g., yellow #5, blue #2,red #40), FD&C lakes; Riboflavin; β-carotene; natural coloring agents, including, for example, fruit, vegetable, and/or plant extracts such as grape, black currant, aronia, carrot, beetroot, red cabbage, and hibiscus.

Exemplary preservatives include sorbate, benzoate, and polyphosphate preservatives.

Suitable emulsifiers include, but are not limited to, diglycerides; monoglycerides; acetic acid esters of mono- and diglycerides; diacetyl tartaric acid esters of mono- and diglycerides; citric acid esters of mono- and diglycerides; lactic acid esters of mono- and diglycerides; fatty acids; polyglycerol esters of fatty acids; propylene glycol esters of fatty acids; sorbitan monostearates; sorbitan tristearates; sodium stearoyl lactylates; calcium stearoyl lactylates; and the like.

Suitable agents for pH adjustment include organic as well as inorganic edible acids. The acids can be present in their undissociated form or, alternatively, as their respective salts, for example, potassium or sodium hydrogen phosphate, potassium or sodium dihydrogen phosphate salts. Exemplary acids are edible organic acids which include citric acid, malic acid, fumaric acid, adipic acid, phosphoric acid, gluconic acid, tartaric acid, ascorbic acid, acetic acid, phosphoric acid and mixtures thereof.

Angiogenin may be present in the food product/nutraceutical formulation in an amount of from about 0.01% to about 50% by weight, e.g., from about 0.01% to about 0.1%, from about 0.1% to about 0.5%, from about 0.5% to about 1.0%, from about 1.0% to about 2.0%, from about 2.0% to about 5%, from about 5% to about 7%, from about 7% to about 10%, from about 10% to about 15%, from about 15% to about 20%, from about 20% to about 25%, from about 25% to about 30%, from about 30% to about 35%, from about 35% to about 40%, from about 40% to about 45%, or from about 45% to about 50% by weight.

Where the food product is a beverage, the food product generally contains, by volume, more than about 50% water, e.g., from about 50% to about 60%, from about 60% to about 95% water, e.g., from about 60% to about 70%, from about 70% to about 80%, from about 80% to about 90%, or from about 90% to about 95% water.

Where the food product is a bar, the food product generally contains, by volume, less than about 15% water, e.g., from about 2% to about 5%, from about 5% to about 7%, from about 7% to about 10%, from about 10% to about 12%, or from about 12% to about 15% water. In some embodiments, the food product/nutraceutical is essentially dry, e.g., comprises less than about 5%, water.

Monosaccharides, disaccharides, and complex carbohydrates, if present, are generally present in an amount of from about 0.1% to about 15%, e.g., from about 0.1% to about 1%, from about 1% to about 5%, from about 5% to about 7%, from about 7% to about 10%, or from about 10% to about 15%, by weight each. Soluble fibers, edible fibers, and emulsifiers, if present, are generally present in an amount of from about 0.1% to about 15%, e.g., from about 0.1% to about 1%, from about 1% to about 5%, from about 5% to about 7%, from about 7% to about 10%, or from about 10% to about 15%, by weight each.

Other components discussed above, if present, are present in amounts ranging from about 0.001% to about 5% by weight of the composition.

The food product or animal feed may further include at least one supplement or treatment, such as bovine somatotrophin, antibiotics, or nutritional supplements. The transgene may optionally comprise one or more of these supplements or they can be administered by other means.

### Research Applications

The subject recombinant microorganisms find use in research, to analyze the effects of angiogenin and its proposed modulators in various tissues. The subject recombinant microorganisms are useful for studying the regulation of muscle synthesis. In particular, the subject recombinant microorganisms are useful for studying the regulation of transcription and translation of angiogenin.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric.

While the invention is specifically described with reference to yeasts, it will be clearly understood that the method of the invention is applicable to a wide range of microorganisms, including organisms used in cheese, buttermilk and yoghurt starter cultures, organisms used in malolactic fermentation, and organisms used in the production of fermented food products such as soya sauce, kimchi, and sauerkraut.

### Example 1

### Generation and Characterization of Recombinant Yeast

### Materials and Methods

An expression cassette capable of expressing angiogenin in transgenic yeast is made using a yeast promoter, an angiogenin coding sequence, and optionally a portion of the MFα1 pre-pro sequence.

For example, the yeast ADHII promoter is obtained from pADR2 (Beier and Young, Nature 300:724-728, 1982) as a SphI fragment of approximately 1530 bp. This fragment is sub-cloned into an M13 phage vector and mutagenised essentially as described by Zoller et al. (Manual for Advanced Techniques in Molecular Cloning Course, Cold Spring Harbour Laboratory, 1983) using a mutagenic primer having the sequence GTA ATA CAC AGA ATT CAT TCC AGA AA. The replicative form of the mutagenised phage is digested with SphI and EcoRI and a partial ADHII promoter fragment of approximately 176 by is isolated. The upstream portion of the promoter is then restored by joining the approximately 176 bp isolated fragment, the approximately 1 kb BamHI-SphI fragment of ADHII (from pADR2), and BamHI-SphI digested phagemid vector pUC13. The resultant vector is designated pUCADH2.

The MFα1 pre-pro sequence is obtained from a yeast genomic library of partial Sau3A fragments cloned into the BamHI site of the yeast expression vector Yep13 (Nasmyth and Tatchell, Cell 19:753-764, 1980). It is identified by complementation of the matα2 mutation. The MFα1 sequence is cut at position −71 with HinfI, the ends filled using DNA polymerase I (Klenow fragment), and EcoRI linkers are added to the ends of the fragment. The MFα1 pre-pro signal sequence is then isolated as an EcoRI-HindIII fragment and sub-cloned into phagemid vector pUC12. The resultant vector is designated pUCMFα1.

HindIII linkers are added to an isolated angiogenin coding sequence selected from the group consisting of SEQ ID NOs: 1-7, before the resultant fragment is digested with HindIII and EcoRV. The digested fragment is then purified and SalI linkers ligated onto the EcoRV terminus. The resultant fragment is then digested with SalI and purified.

The pUCMFα1 vector is then digested with PstI and HindIII to isolate the approximately 237 by MFα1 pre-pro sequence fragment. This fragment is then joined to the purified angiogenin fragment before both are ligated into PstI-SalI digested phagemid vector pUC13. The entire MFα1-angiogenin fragment is then isolated by digestion with PstI and SalI and inserted into PstI-SalI digested M13mp10 (replicative form). A precise junction between the Lys-Arg processing site of MFα1 and the first amino acid of angiogenin is achieved by in vitro mutagenesis of the resultant recombinant phage, using the mutagenic primer TGG ATA AAA GAC AGG ATA ACT C. The replicative form of the mutagenised phage is digested with PstI and SalI to release the MFα1-angiogenin fragment. This fragment is then purified in readiness for assembly of the expression cassette.

To assemble the final expression cassette, pUCADH2 is digested with BamHI and EcoRI and the resulting ADHII fragment is purified. The vector pUCMFα1 is then digested with EcoRI and HindIII and the resulting MFα1 fragment is purified. These two fragments are then ligated into BamHI-HindIII digested pUC12. The resulting vector is then digested with BamHI and PstI to release the ADHII-MFα1 fragment. The purified ADHII-MFα1 fragment is then ligated to the MFα1-angiogenin fragment, and BamHI-SalI digested pUC12, in a triple ligation. The resulting vector comprises the angiogenin expression cassette in a pUC12 backbone.

The angiogenin expression cassette is then transferred to the yeast expression vector YEp13 by BamHI-HindIII digestion of both the angiogenin expression cassette in the pUC12 backbone and the YEp13 expression vector, followed by ligation of the fragments and selection of YEp13 vectors containing the desired insert. The resultant yeast expression vector comprising the angiogenin expression cassette is designated YE13-ADHII-MFα1-angiogenin.

The YE13-ADHII-MFα1-angiogenin expression vector is then used to transform Saccharomyces cerevisiae yeast cells, which are cultured by conventional methods to express the angiogenin transgene. Such culture methods include aerobic fermentation with 36ATP/glucose and anaerobic fermentation with 2ATP/glucose.

While the above example utilises Saccharomyces cerevisiae, it is contemplated that other yeasts, such as Pichia spp., and lactic acid bacteria, such as Lactobacillus spp., may also be suitable for use in the expression of angiogenin. There are many commercially available yeast expression vectors and the selection of an appropriate vector for use in the species of yeast or lactic acid bacteria being used could be made by one of skill in the art.

It is further contemplated that the promoter used in the expression vector may be either constitutive or inducible. If an inducible promoter is used, then the agent to be used for induction of expression must be suitable for human and/or animal consumption as it may potentially be incorporated into the food, feed or food additives made using the transgenic yeast. An example of a suitable induction agent would be galactose.

It is contemplated that the angiogenin expressed by the transgenic yeast may be secreted into the culture medium, or it may be contained within the yeast cells.

Assays can be performed to ascertain the amount of angiogenin produced by the transgenic yeast cells. Such assays would involve the use of Protein gel electrophoresis, Western blotting, ELISA and or HPLC analysis of the yeast culture medium and yeast cell extracts. A biotinylated Anti-human Angiogenin Antibody for use in ELISA assays for angiogenin is available from R&D Systems (Catalogue #BAF265).

The angiogenin produced by the transgenic yeast may be used as a food, feed, or food additive, for both animals and humans. In addition, the transgenic yeast may itself be used in the production of a food or feed for humans or animals, whereby the human or animal consumes the angiogenin when consuming the food made with the transgenic yeast. For example, the angiogenin expressing transgenic yeast may be used in the production of fermented food products such as yoghurt, cheese, salami, probiotic foods, breads, wine, beer, cider, aquaculture animal feeds, and other animal feeds.

The angiogenin may also be purified from the transgenic yeast. Techniques for the purification of the angiogenin produced by the transgenic yeast include capture by cation exchange as outlined in PCT/AU2007/001719, further purified by affinity chromatography as outlined in PCT/AU2009/000604, and then desalt using either ultra-filtration or size exclusion chromatography.

1. A recombinant microorganism comprising a transgene encoding angiogenin.

2. The recombinant microorganism of claim 1 in which the transgene further encodes follistatin.

3. The recombinant microorganism of claim 1 further comprising a transgene encoding follistatin.

4. The recombinant microorganism of claim 1 in which the transgene further comprises a signal sequence for a secreted polypeptide or protein and optionally a specific protease cleavage site between the signal sequence and angiogenin.

5. An expression cassette comprising a coding sequence for angiogenin operably linked to a heterologous promoter.

6. A host cell, transformed with the expression cassette of claim 5.

7. A method for producing a recombinant microorganism comprising an angiogenin transgene comprising fermenting the host cell of claim 6 in growth medium.

8. The method of claim 7 in which the recombinant microorganism secretes the angiogenin into the growth medium.

9. The microorganism of claim 1, in which the microorganism is a yeast, yeast of a genus Candida, Debaromyces, Saccharomyces, Pichia, Hansenula, Kluyveromyces, Saccharomyces cerevisiae, Pichia pastoris, Saccharomyces cerevisiae strains suitable for production of beer, wine, or bread, a lactic acid bacterium, a probiotic organism, a lactic acid bacterium of the homolactic or heterolactic fermentation type, Lactobacillus species, Lactobacillus acidophilus, amylovorus, brevis, bulgaricus, buchneri, casei, confusus, crispatus, cucumeris, curvatis, delbrueckii, farciminis, fermentum, fructivorans, gasseri, helveticus, hilgardii, johnsonii, kefiri, lactis, leichmanii, lotus, paracasei, pasterianus, pentosus, plantarum, rhamnosus, reuteri, pentoaceticus, plantarum, sakei, salivarus, and sanfranciscensis; Bifidobacterium species, Bifidobacterium adolescentis, animalis, bifidum, infantis, lactis, longum, pseudolongum and breve; Leuconostoc species, Leuconostoc cremoris, lactis, mesenteroides, mesenteroides var. Sake, and onei; Micrococcus species; Pediococcus species, Pediococcus acidilatici, cerevisiae, halophilus, homari, pentosaceus and soyae; Propionibacterium species, Propionibacterium acidipropioici, arabinosum, freudenreichii, shermani and thoenii; Acetobacter species, Acetobacter rancens and xylium; Bacillus species, Bacillus brassicae fermentati, citreus, laterosporus, coagulans, licheniformis, natto and pumillus; Clostridium species, Clostridium bifermentans; Corynebacterium species, Corynebacterium kusaya, Halobacterium species, Halococcus species, actococcus species, actococcus lactis and cremoris; Enterococcus species, Enterococcus durans and faecium; Enterobacter aerogenes; Staphylococcus species, Staphylococcus aureus, carnosus, equorum, sciuri, xylosus, epidermidis, and Streptococcus species, Streptococcus cremoris, lactis, lactis var. diacetylactis, lactis var. hollandicus faecalis, and thermophilus.

10. A method of production of a fermented food or beverage product, comprising the step of culturing a culture comprising the recombinant microorganism of claim 1.

11-12. (canceled)

13. A food product, beverage product or animal feed produced from or comprising the recombinant microorganism of claim 1.

14. A method of treating muscle disorders, muscle wasting disorders, muscular dystrophy, muscular atrophy, sarcopenia, cachexia, improving muscle form by improving muscle strength, mass or exercise tolerance, decreasing fat, improving muscle to fat ratio, treating diseases caused by or involving suboptimal muscle to fat ratio which effect is enhanced by follistatin, treating bone disorders, osteoporosis, improving bone density, treating neurological disorders or diseases affecting the nervous system, and treating motor neurone diseases, ALS, spinal muscular atrophys, inflammation myopathies, dermatomyositis, polymyositis, inclusion body myositis, diseases of the neuromuscular junction, Myasthenia Gravis (MG), Lambert-Eaton Syndrome (LES), Congenital Myasthenic Syndrome (CMS), myopathies due to endocrine abnormalities, Hyperthyroid Myopathy (HYPTM), Hypothyroid Myopathy (HYPOTM), diseases of peripheral nerve, Charcot-Marie-Tooth Disease (CMT), Dejerine-Sottas Disease (DS), Friedreich's Ataxia (FA), other myopathies, Myotonia Congenita (MC), Paramyotonia Congenita (PC), Central Core Disease (CCD), Nemaline Myopathy (NM), Myotubular Myopathy (MTM or MM), Periodic Paralysis (PP), metabolic diseases of muscle, Phosphorylase Deficiency (MPD or PYGM), Acid Maltase Deficiency (AMD), Phosphofructokinase Deficiency (PFKM), Debrancher Enzyme Deficiency (DBD), Mitochondrial Myopathy (MITO), Carnitine Deficiency (CD), Carnitine Palmityl Transferase Deficiency (CPT), Phosphoglycerate Kinase Deficiency (PGK), Phosphoglycerate Mutase Deficiency (PGAM or PGAMM), Lactate Dehydrogenase Deficiency (LDHA), Myoadenylate Deaminase Deficiency (MAD), diseases connected to impaired lipid metabolism, dyslipidemia, related lipid abnormalities, hyperlipidemia, hypercholesteremia, hypertriglyceridemia, mixed dyslipidemia, spine injuries or diseases, diseases involving glucose homeostasis, for providing neuroprotection, nervous system functional support, managing metabolic diseases and diseases connected to impaired glucose metabolism and impaired insulin action, diabetes mellitus, diabetes mellitus type 1 and 2, non-autoimmune non-insulin dependent diabetes mellitus, syndrome X or metabolic syndrome, for providing microbial inhibition and enhancing gut epithelial function, for wound healing and bacterial flora symbiosis, for promoting gut health and gut based disease prevention and immune enhancement, for improvement of animal health including gut/immune function, and muscle mass and muscle to fat ratio to provide improved carcass composition, by administering to a subject in need thereof a therapeutically effective amount of angiogenin and optionally follistatin derived from the recombinant microorganism of claim 1 or the food product, beverage product or animal feed of claim 13 or an extract thereof comprising angiogenin.
